(19) United States
(12) Patent Application Publication
(10) Pub. No.: US 2024/0355470 A1
(43) Pub. Date: Oct. 24, 2024

(54) SYSTEM FOR CONDITION TRACKING AND MANAGEMENT AND A METHOD THEREOF

(71) Applicant: Ocean Friends LLC, Irving, TX (US)

(72) Inventors: Animesh Kumar, Irving, TX (US); Manish Shukla, North Bend, WA (US); Kushagra Mittal, Delhi (IN); Anusha Tiwari, New Delhi (IN)

(21) Appl. No.: 18/305,675

(22) Filed: Apr. 24, 2023

(51) Int. Cl. G16H 50/20; G06F 40/30; G16H 10/60; H04L 67/306

(52) U.S. Cl. CPC G16H 50/20; G06F 40/30; G16H 10/60; H04L 67/306

(57) ABSTRACT

A system and method for condition tracking and management is disclosed. In the method, a user input including a message related to manifestation of the condition is received by a user. Based on one or more condition tracking parameters, condition related information is extracted from the user input. The condition related information is associated with the user. The condition related information is then classified to one or more disease tracking parameters by allocation the information to the corresponding condition tracking parameters. Further, the condition related information is compared to pre-stored condition related information, by comparing a user profile of the user with one or more other user profiles that are pre-stored. Subsequently, a response including one or more recommendations appropriate to the at least one user input is generated and the response is provided to the user.

### TECHNICAL FIELD

The present disclosure relates to systems and related methods for tracking and management of a condition. In particular, the disclosure relates to tracking a condition and providing appropriate recommendations to a user that aid in management of the condition.

### BACKGROUND

A rare disease is a condition that affects a small portion of the population. There are many different types of rare diseases including genetic disorders and autoimmune diseases such as Autism, Alzheimer's, Parkinson's and so on. Some rare diseases are caused by a single genetic mutation, while others may have multiple genetic and environmental factors that contribute to their development. Despite affecting a small number of people, there are over 7,000 rare diseases, and together they affect approximately 400 million people worldwide. Based on one of the recent CDC reports, 1 in 26 or approximately 2.7% of 8-year-olds that are born in US have Autism. Many of these diseases are chronic and life-threatening, and often there are no FDA-approved treatments or cures. This can make it difficult for individuals with rare diseases and their families to access the care and support they need.

Some diseases such as Alzheimer's and Autism are so rare that when a patient suffers from such diseases, the caregivers fail to understand its manifestation. Autism, also known as Autism Spectrum Disorder (ASD), is a neurodevelopmental disorder that affects communication, social interaction, and behavior. People with autism often experience difficulties in interpreting social cues, making friends, and engaging in typical back-and-forth conversations. They may also exhibit repetitive behaviors, strict routines, and intense interest in specific subjects. The autism spectrum is a range of conditions, and each person with autism is unique, with his or her own strengths and challenges. Some people with autism are highly skilled in areas such as mathematics, music, or art, while others may require significant support to meet their daily needs. While there is currently no cure for autism, early diagnosis and appropriate treatment can make a significant difference in the lives of individuals with autism and their families.

Parents of children with autism can face a number of challenges, both practical and emotional. Some of these challenges include diagnosis and early intervention since getting a proper diagnosis for a child with autism can be a long and confusing process, and finding early intervention services can be difficult; and limited social support as it can be hard for parents of children with autism to find other families who understand what they're going through. Some families may feel isolated as they don't have anyone to turn to for support. It's important for parents of children with autism to seek out support from a variety of sources, including family and friends, support groups, and mental health professionals. Having a strong support network can help families navigate the challenges of raising a child with autism and ensure that their child receives the best possible care.

Building a support network is a crucial aspect of addressing the challenges faced by parents of children with autism. A support network can provide a sense of community, a place to turn to for advice and support, and can help alleviate feelings of isolation and loneliness. Joining a support group specifically for parents of children with autism can be particularly beneficial. This can be a group that meets in person or an online community. These groups can provide a safe space for parents to share their experiences, ask questions, and connect with others who understand the unique challenges they face. In addition to support groups, parents of children with autism can also connect with other families of children with autism, either through local events, advocacy organizations, or online forums. Building a network of families who are also navigating similar challenges can help provide a sense of community and a source of support and advice.

It is also important for parents of children with autism to seek out support from mental health professionals, such as therapists or counselors. This can help them manage stress, anxiety, and depression, and provide a space to process their experiences and emotions. Having a strong support network can be instrumental in helping families navigate the challenges of raising a child with autism and ensuring that their child receives the best possible care. By reaching out for help and building a network of support, parents of children with autism can find the resources and support they need to thrive.

Online platforms can be a great resource for parents of children with autism. There are several online communities, websites, and forums dedicated to providing support and information for families of children with autism. For example, some popular websites include Autismspeaks.org which is a website that provides information and resources for families affected by autism, including a directory of support groups and an online community forum; Wrongplanet.net, a website and online community specifically for individuals with autism and their families. It provides a forum for discussion, support, and information exchange; Numerous Facebook groups are dedicated to providing support and resources for families of children with autism. Some of these groups are specifically for parents of children with autism, while others are for individuals on the autism spectrum and their families; and Online support groups are for families of children with autism. These groups provide a virtual space for parents to connect, share their experiences, and offer support to one another. By participating in these online communities, parents of children with autism can connect with others who understand the unique challenges they face, find support and encouragement, and access a wealth of information and resources.

However, the shortcoming of these existing websites and groups is that the users need to visit them time and again. Also, the users need to find people who can answer their questions, which is time-consuming and might not be accurate for their specific needs. Also, there is no provision of making an exhaustive profile that can be used to make personalized recommendations. Moreover, enrolling on so many platforms and groups require the users to fill details each time which is time consuming and not user friendly.

Therefore, there is a need for a system and method that can provide individualized care plan to patients. Also, the system should make recommendations based on unique manifestation of the condition in each patient. Furthermore, the system should be easy to use by the parents, guardian and caregiver of the patients.

### SUMMARY

The present disclosure related to a system and method for tracking and management of a condition in a patient. The present disclosure addresses a need to track the manifestation of condition in the patient. The present disclosure also provides a response including one or more recommendations for management of condition in the patient. The present disclosure discloses a method for receiving user inputs related to manifestation of the condition and generating response appropriate to manage the condition in the patient.

In one aspect of the present disclosure, a method for condition tracking and management is disclosed. According to the disclosed method, a user input including a message related to manifestation of the condition is received by a user. Based on one or more condition tracking parameters, condition related information is extracted from the user input. The condition related information is associated with the user. The condition related information is then classified to one or more condition tracking parameters by allocation the information to the corresponding condition tracking parameters. Further, the condition related information is compared to pre-stored condition related information, by comparing a user profile of the user with one or more other user profiles that are pre-stored. Subsequently, a response including one or more recommendations appropriate to the at least one user input is generated and the response is provided to the user. In some aspects, the method further includes an input provided by an expert, where the input is used in generating the response and the expert may be a physician or any person having experience in providing recommendations relevant to management of the condition.

In another aspect of the present disclosure, a computer system for condition management is disclosed. The system includes a server and a processing device that is operatively coupled to the server. The server further includes a memory to store instructions, while the processing device executes the instruction to: receive a user input, where the user input includes a message related to manifestation of the condition in a user; extract, based on one or more condition tracking parameters, condition related information from the user input, where the condition related information is associated with the user; classify the condition related information to the one or more condition tracking parameters, by allocating the one or more condition related information to the one or more corresponding condition tracking parameters; compare the condition related information to pre-stored condition related information, by comparing the user profile of the user with one or more other user profiles that are pre-stored, to generate a response appropriate to the at least one user input; and provide the response including one or more recommendations to the user.

In one aspect, the processing device of the above discussed computer system includes a bot that is configured to share the received user input with a recommendation generating module, where the bot and the recommendation generating module are operatively coupled. The recommendation generating module processes the received user input using a data model to generate a response corresponding to the received input. In one aspect, the user interface is integrated with the bot and is configured to receive the user input related to manifestation of the condition in the patient. For instance, the user interface can be a WhatsApp application having a bot integrated therein. In this scenario, the user can open the WhatsApp application and provide his input related to the condition and the bot integrated with the WhatsApp application shares the received user input with the recommendation generating module for further processing.

In one aspect, the recommendation generating module compares the user profile with other user profiles that are pre-stored, to generate a response appropriate to the received user input. Further, the response is provided to the user that includes one or more recommendations related to management of the condition.

In one aspect of the present disclosure, the user input is related to one or more condition-related parameters, including parameters related to sleep, diet, behavior, speech, intervention and default. For instance, the user input may relate to patient's symptoms such as episodes of hyperactivity, migraine, temper tantrum or the like. Further, speech related user input can include type of words and sounds made by the patient, intervention related inputs can be medications or any over-the-counter drugs taken by the patient on a particular day and time.

In yet another aspect, the user interface may include a messaging application including WhatsApp, Facebook Messenger, SMS, Alexa, Siri, Google Voice and so on.

In yet another aspect, the processing device includes a handheld device, a mobile device, a voice enabled device, or a wearable device.

In yet another aspect, the server includes a memory configured to store instructions which includes the user inputs and the recommendations generated in response to the received inputs.

Advantageously, the user interface may include a graphical user interface, a voice user interface, a gesture user interface, or any suitable user interface. Also, the user may provide a text input, a voice command, or a gesture as an input.

### BRIEF DESCRIPTION OF THE ACCOMPANYING DRAWINGS

The above-mentioned implementations are further described herein with reference to the accompanying figures. It should be noted that the description and figures relate to exemplary implementations and should not be construed as a limitation to the present disclosure. It is also to be understood that various arrangements may be devised that, although not explicitly described or shown herein, embody the principles of the present disclosure. Moreover, all statements herein reciting principles, aspects, and embodiments of the present disclosure, as well as specific examples, are intended to encompass equivalents thereof.

FIG. 1 illustrates a system used for condition tracking and management, in accordance with one implementation of the present disclosure.

FIG. 2 illustrates an example implementation using data model for tracking and management of condition in a patient, in accordance with one implementation of the present disclosure.

FIG. 3 depicts an example implementation disclosing working of the data model for tracking and management of condition in a patient, in accordance with one implementation of the present disclosure.

FIG. 4 illustrates an example method for tracking and management of condition in a patient by disclosing use of default category, in accordance with one implementation of the present disclosure.

FIG. 5a illustrates an example method tracking behavior of a user to provide an appropriate response for management of the condition, in accordance with one implementation of the present disclosure.

FIG. 5b illustrates another example method tracking behavior of a user to provide an appropriate response for management of the condition, in accordance with one implementation of the present disclosure.

FIG. 6 illustrates a method for tracking and management of condition in a patient, in accordance with one implementation of the present disclosure.

### DETAILED DESCRIPTION

Embodiments, of the present disclosure, will now be described with reference to the accompanying drawing.

In the following description, certain specific details are set forth in order to provide a thorough understanding of various disclosed embodiments. However, one skilled in the relevant art will recognize that embodiments may be practiced without one or more of these specific details, or with other methods, components, materials, etc.

Unless the context indicates otherwise, throughout the specification and claims which follow, the word “comprises” and variations thereof, such as, “comprises” and “comprising” are to be construed in an open, inclusive sense that is as “including, but not limited to.” Further, the terms “first,” “second,” and similar indicators of the sequence are to be construed as interchangeable unless the context clearly dictates otherwise.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its broadest sense, that is, as meaning “and/or” unless the content clearly dictates otherwise.

The systems and methods disclosed herein includes receiving at least one user input, where the user input includes a message related to manifestation of the condition in a user. The systems and methods analyze the user input using a data model (such as a contextual large language model) for tracking the manifestation of the condition and/or making recommendations appropriate in management of the condition. In addition, the systems and methods provide personalized recommendations to the caregivers of the patients suffering from subject condition, based on profile tracking. While the description presented herein makes a specific reference to Autism, it is to be appreciated that aspects of the present disclosure are also equally applicable to other rare condition such as Dementia, Alzheimer's, Parkinson, or any other diseases.

The present disclosure, in one preferred embodiment, includes a computer system for condition tracking and management. In such implementation, the system receives a user input which includes message related to manifestation of the condition in a user. In cases, where the user is interacting with the system for a first time, the system creates a user profile of the patient. The user profile includes information related to the patient that aid in tracking manifestation of the condition in the patient. For instance, the information can include communication and social interaction of the patient, his repetitive behaviors and interests, sensory processing, adaptive skills, behavioral changes, health parameters and so on. However, it should be noted that the information may include other information related to the patient such as his age, gender, location, height, weights, and so on. Further, the user profile also stores information related to a device used for providing user inputs, for instance, mobile number, email id, IP address, and so on.

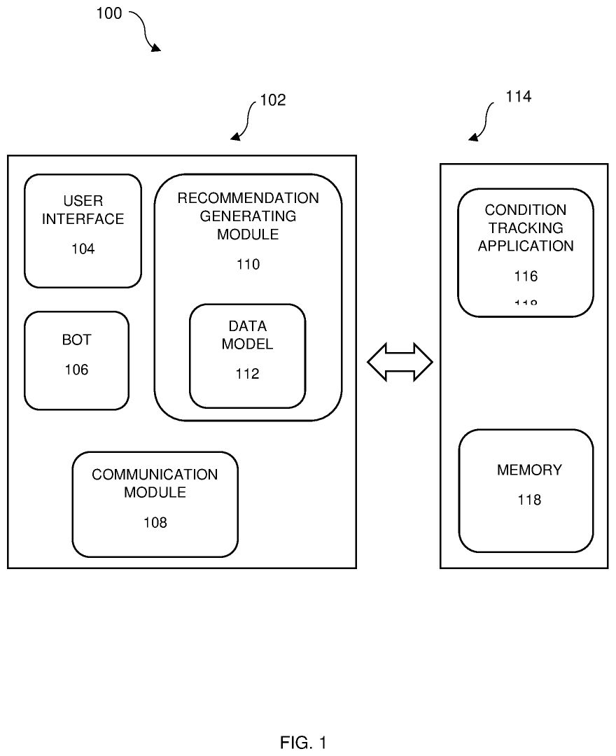

Referring now to FIG. 1, a system 100 used for condition tracking and management in a patient is disclosed, in accordance with one implementation of the present disclosure. While the system 100 discussed here refers to condition tracking and management, it should be noted that the system 100 may also be used for other tracking applications including, but is not limited to, fitness tracking, activity tracking, employee activity tracking, daily activity tracking, time tracking, and the like.

The system 100 includes one or more components that work in tandem to provide a seamless user experience. In the shown embodiment, the system 100 includes a processing device 102 that is communicatively coupled to a server 114. As shown, the processing device 102 includes a user interface 104, a bot 106, a communication module 108, a recommendation generating module 110, and data model 112, while the server 114 includes a condition tracking application 116 and a memory 118.

The processing device 102 serves as a primary interface between the user and the system 100. The user interface 104 is integrated with the bot 106 such that the user interface allows the user to interact with the bot 106. The user interface 104 is designed to be intuitive and easy to use, enabling the user to communicate with the bot 106 without any difficulty. The user interface 104 and the bot 106 are integrated to each other and is configured to receive user inputs. In some scenarios, the user input may be related to manifestation of the condition in a user. However, in other scenarios, the user input may be related to any information that is not directly related to manifestation of the condition in the user. Hereinafter, the term ‘user input’ may be interchangeably referred to as ‘condition related input’, ‘input’, ‘user inputs’, without deviating from the scope of the present disclosure.

The system 100 disclosed herein allows the users to provide inputs through commonly used user interfaces 104 (such as WhatsApp, Siri, Alexa, Google Home, SMS), thereby removing any hurdle of using a new application. Moreover, the user can provide his inputs directly to the bot 106, and hence the user is not required to install any new application on the processing device 102.

The system integrates the bot 106 with the user interface 104 to receive user input in a seamless manner. For instance, the user may simply open his/her WhatsApp application and input any information related to the condition. The system allows contextual analysis of the user inputs using fine-tuned large language models. Such analysis allows extraction of condition related information from the user input based on condition tracking parameters such as symptoms, diet, sleep, intervention, behavior, speech and so on. Further, analysis of the condition related information allows the recommendation generating module 110 to provide a response that is appropriate to the received user input and aid in condition management. Further details pertaining to the analysis of condition related information will be discussed in subsequent section.

The processing device 102 of the system 100 can be any device that is equipped with the necessary components to interact with the user, receive inputs, process inputs, and communicate with other system components for tracking and management of the condition in the user. Some examples of such devices include smartphones, tablets, personal computers, and so on. With its advanced technology and user-friendly interface 104, the processing device 102 can provide an ideal platform for the user to interact with the system 100 and manage their condition.

It should be noted that the processing device 102 can be a wearable device such as a smartwatch or a fitness tracker. With their compact form factor and convenient location on the user's wrist, such wearable devices can provide an accessible and easy-to-use platform for the user to manage their condition. In some scenarios, the processing device 102 can be a voice-enabled device that can receive user inputs in the form of a voice command and thus provide voice recommendations based on received user inputs. The processing device 102 that is used in the system 100 may vary based on user's preferences and requirements. To that end, the processing device 102 may be a handheld device, a mobile device, a voice enabled device, or a wearable device.

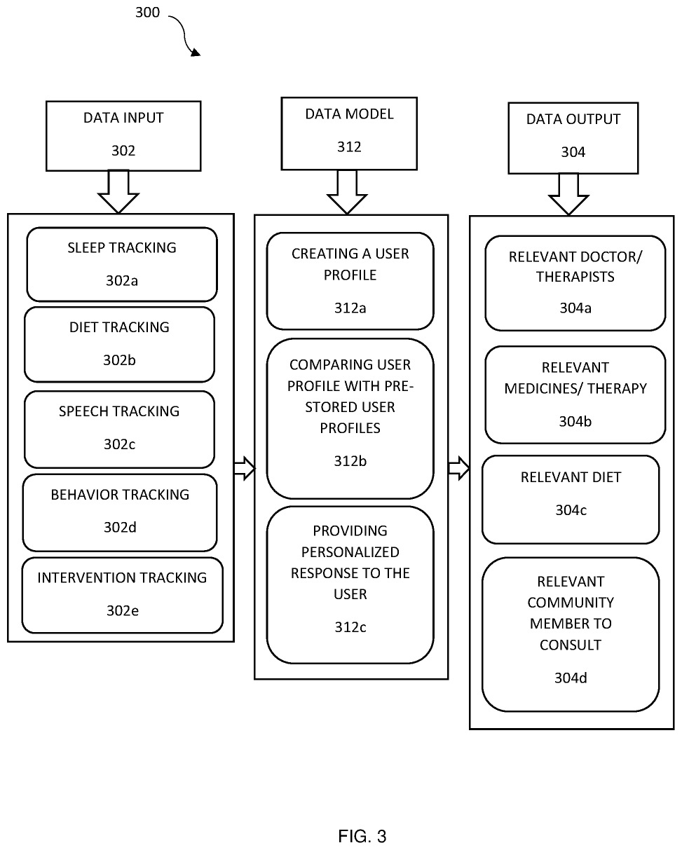

Further, the communication module 108 enables the processing device 102 to communicate with the condition tracking application 116. The communication module 108 ensures that the processing device 102 can receive and transmit data from and to the condition tracking application 116, thereby providing the user with a comprehensive view of their condition status. As shown in FIG. 3 and will be discussed in detail later, the condition tracking application 116 may be communicatively coupled to a processor (not shown in the figure) that processes the received user inputs and generates a response based on the received inputs.

The recommendation generating module 110 is communicatively coupled to the bot 106 such that any user inputs received by the bot 106 are processed by the recommendation generating 110. In the shown embodiment, the recommendation generating module 110 includes the data model 112 and is configured to process the user inputs by extracting condition related information from the input, based on one or more condition tracking parameters (for example, sleep, diet, behavior, speech and intervention). The condition related information is associated with the user and the condition related information is further classified to one or more condition tracking parameters, by allocating the condition related information to the one or more corresponding condition tracking parameters. The extracting of condition related information includes identifying the intent of the message through NLP (natural language processing) of the user input. The classification of condition related information is done based on the corresponding condition tracking parameters included in the user input. Such condition tracking parameters may include one or more of sleep, diet, speech, behavior, intervention, and default. For instance, if the user input is “I slept at 9 PM and woke up at 6 AM”, the condition related information in this message is related to sleep and thus the extracted information is classified under ‘sleep’ tracking category. Further, if the user enters “I had juice in breakfast”, the information relates to the breakfast type and time, thereby the information will be classified to the ‘diet’ tracking category. Similarly, the system 100 classifies any other user input into relevant condition tracking parameters. In some scenarios, where the user input is not related to any of the defined condition tracking parameters, the input message is classified under ‘default’ category. In such scenarios, the user receives a response message asking him/her to enter a valuable input, which indicates that the input message should be related to one or more of the defined condition tracking parameters. One example of such input message that may be classified under default include “Will you help me in tracking my condition”.

The system 100 classifies the disease related information to the condition tracking parameters based on contextual analysis of the user input. Such analysis identifies a correlation of the user input to the one or more condition tracking parameters and also identify an intention of the user behind the received user input.

The recommendation generating module 110 is configured to generate a response based on comparison of the condition related information to pre-stored condition related information. This may include comparing the user profile with one or more other user profiles that are pre-stored. The generated response may include one or more recommendations appropriate to the user input, where the recommendations aid in management of the condition in the patient. The recommendations generated may include relevant diet, relevant therapy/medicine, details of relevant hospital/doctor/therapists, details of relevant community member whose profile matches with the user profile, and so on.

More details pertaining to the types of user inputs and the processing of these inputs to provide relevant recommendations are discussed in FIG. 3.

In some scenarios, the system 100 compares profile of the user with other users based on the user input. Based on this profile comparison, the system 100 helps in predicting symptoms and journey of the patient by comparing it with the profile of other users who witnessed similar symptoms early in their condition manifestation journey.

Further, the response is provided to the user that includes one or more recommendations related to management of the condition. The response is provided by ranking a plurality of recommendations such that the recommendations ranked higher are likely to be provided as the one or more recommendations. In one scenario, the recommendations are ranked based on their effectiveness scores such that the recommendation having a higher score is ranked higher as compared to the recommendation with lower effectiveness score. In addition, the generated response nay also includes an expert input, where the expert may be a physician or an expert who can manage the condition well. The expert input may be pre-stored or can be provided in real-time. Therefore, the response generated and provided to the user is vetted by the expert and hence is more reliable and effective in managing the condition.

Further, the system 100 includes the communication module 108 that acts as an intermediary between the recommendation generating module 110 and the user, enabling the bot 106 to provide the personalized recommendation to the user via the user interface 104. The communication module 108 may use a variety of communication technologies for providing the processed data to the user, such as text messages, push notifications, or voice responses, depending on the user input.

In the example implementation, the communication module 108 enables the user to receive the personalized recommendations generated by the recommendation generating module 110 in real-time. The quality and reliability of the communication module 108 can have a significant impact on the user experience, and it is important to carefully consider the design of the communication module 108 when building the system 100.

To that end, the communication module 108 should be designed to be flexible and adaptable, allowing the bot 106 to transmit the personalized recommendations to the user in the format that is most convenient for the user, and in a way that is easily accessible and understandable. The communication module 108 should also be designed to be secure, ensuring that the user's information and data remain confidential and protected.

Referring back to the user interface 104, in shown implementation, the bot 106 is integrated with a user interface 104 which may include messaging application such as WhatsApp, Facebook Messenger, SMS, and so on. In such implementation, the bot 106 receives user input related to a condition via the user interface 104 which includes messaging application, and the recommendation generating module 110 then processes the received inputs using data model 112 and generates personalized response based on the received user input. This way the integrated bot 106 is configured to provide the user with a personalized and efficient way to manage their health.

The bot 106 provides personalized response to users based on their inputs related to a condition, thereby delivering a convenient and accessible way to manage their health. The user input includes at least one of text input, audio input, video input and image input.

Typically, integrating the bot 106 the processing device 102 depends on one or more factors including type of processing device 102 and the mode of interaction between the user and the processing device 102. For instance, integrating the bot 106 with voice-enable device such as Amazon Alexa may differ from its integration with applications such as WhatsApp, and so on.

For instance, to integrate the bot 106 with Alexa, it may be needed to create an Alexa skill using the Alexa Skills Kit (ASK) which allows developers to build custom capabilities for Alexa. The bot 106 and Alexa skill can then be connected using a web service, such as AWS Lambda or a REST API, which will handle the processing of requests and responses between the bot and Alexa. Furthermore, to integrate a bot 106 with WhatsApp, one may use the WhatsApp Business API, which allows businesses to send and receive messages to and from the users on WhatsApp. The bot 106 can interact with the WhatsApp Business API using a REST API, and the API will handle the processing of messages between the bot 106 and WhatsApp.

Further, the data model 112 refers to the part of the system 100 that is responsible for processing the inputs received from the user. The data model 112 takes the inputs related to the condition and extracts and classifies them based on condition related information from the user input and generates personalized recommendations for the user using the recommendation generating module 110. This may involve use of machine learning algorithms or other forms of artificial intelligence to analyze the inputs, access relevant databases and knowledge sources, and generate an appropriate response. The data model 112 is a crucial component of the system 100, as it determines the behavior of the bot 106 and the quality of the personalized recommendations that it provides to the user. The data model 112 should be designed to provide accurate, relevant, and helpful recommendations to the user in real-time. In essence, the data model 112 is responsible for classifying the appropriate condition tracking parameter and the recommendation generating module 110, determines the appropriate personalized response to the inputs received from the user. The data model 112 disclosed herein is scalable, efficient, and robust, so that it can handle a large number of inputs and provide quick and accurate responses to the user.

It should be noted that there are several ways to send information received from a bot 106 to the backend server 114 or condition tracking application 116, depending on the platform and tools used in building the bot 106. Some of the approaches that may be implementing for sending such information include webhooks, API calls, middleware, TWILIO and so on.

Further, it should be noted that in some variations, the condition tracking application 116 can be installed on the processing device 102. In such implementation, the condition tracking application 116 may include functionalities of the recommendation generating module and communicates with the remote server 114 for storing the user inputs and corresponding response made thereon.

However, in shown embodiment, the condition tracking application 116 is installed on the server 114. In this scenario, the processing device 102 communicates with the server 114 via a communication network. The server 114 acts as the backbone of the system 100, which stories the condition related information along with user inputs and generated responses in the memory 118. Also, in this scenario, a user profile is created whenever a new user interacts with the bot 106 for a first time. The user profile includes information related to the user related, device related information, user inputs, or generated responses. It may also be noted that in such scenario, the all such information is stored in the memory 118 for any future use.

The system 100 for condition tracking and management is designed to provide individuals with an effective way to manage their condition. The natural language processing technology used by the system 100 ensures that the user inputs are understood and processed accurately, thereby providing the user with relevant recommendations and support.

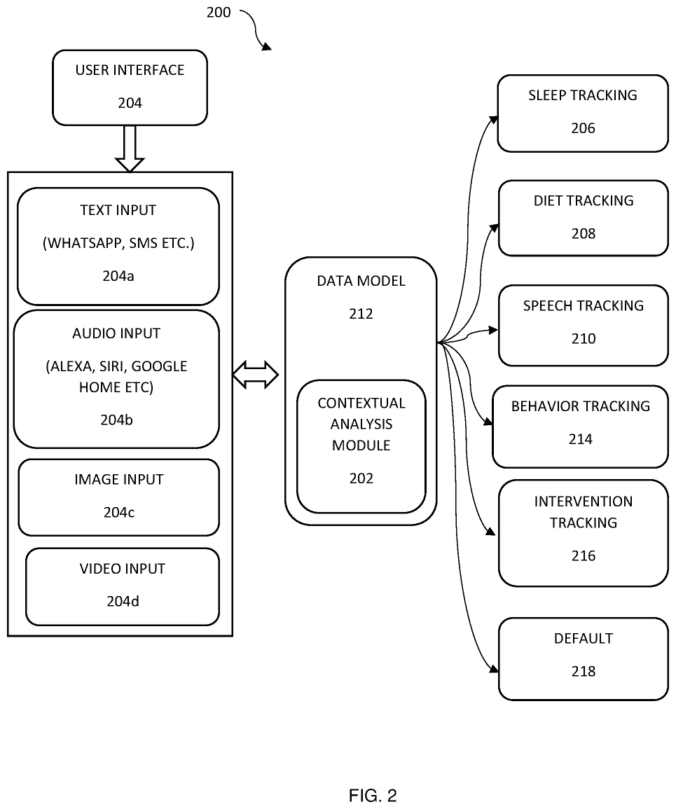

FIG. 2 shows an example implementation 200 using a data model for tracking and management of a condition in a patient, in accordance with one implementation of the present disclosure. As shown, the implementation 200 includes integration of an existing user interface 204 with a data model 212. Here, the user interface 204 may include a graphical user interface, a voice user interface, a gesture user interface, or any suitable user interface. Accordingly, a user may provide a text input, a voice command, or a gesture as an input. For instance, the user may provide a text input 204a via WhatsApp, an audio input 204b or voice input via Google Home, Siri, Alexa, an image input 204c, or a video input 204d.

The data model 212 receives the user inputs. The data model 212 is configured to identify the intent behind the user's input. To that end, the data model 212 further includes a contextual analysis module 202 for analyzing the received user inputs. The contextual analysis module 202 along with the model 212 attempts to extract condition related information from the received inputs, which aid in understanding what the user is trying to convey. For example, if a user inputs a sentence containing profanity, the model may attempt to rephrase the sentence to remove the profanity, or may ask the user to provide more information to clarify his/her intent.

When generating an appropriate response corresponding to received inputs, the data model 212 may use a combination of factors, such as the user inputs, his/her previous behavior or inputs, and other contextual information, in order to provide the response that is more appropriate and contextual the user inputs. However, if the user's input is crude or inappropriate, the model may choose to disregard that input or provide a response that encourages the user to rephrase his/her input in a more appropriate manner.

For instance, the user input may include information about his diet schedule. The data model 212 interprets the inputs and stores the contextual output under diet tracking 208. In another example, the user may provide inputs related to his recent sleep pattern. For instance, the user may provide a text input via WhatsApp having integrated bot 106 saying “I had trouble sleeping till lam last night”. The data model 212 may interpret the inputs and stores the input under sleep tracking 206 as “disturbed sleep” on that particular date. In addition, the data model 212 may interpret the input as a request to provide recommendations for tonight if the same sleep pattern repeats. Accordingly, the model 212 may provide recommendations related to sleep hygiene techniques, relaxation techniques before going to bed, and over-the-counter sleep aids if nothing helps, and so on.

Similar to above, the data model 212 analyzes each input and interpret if the input should be stored under sleep tracking 206, diet tracking 208, speech tracking 210, behavior tracking 214, intervention tracking 216 or default 218.

Further, the data model 212 can learn and improve over time, as it gets exposed to more user inputs, response, and other related data. The data model 212 is trained using large amounts of data and sophisticated machine learning techniques, which enables the data model 212 to learn patterns and relationships within the data. It provides response to the user by ranking a plurality of recommendations such that the recommendations ranked higher are likely to be provided as the one or more recommendations. Further, the data model 212 gets updated each time the user provides a new user input or a corresponding response is generated.

The data model 212 disclosed is configured to learn and adapt to new inputs and feedback from users. For example, if users consistently provide positive feedback for certain responses or recommendations, the data model 212 may learn to prioritize those options in the future. Similarly, if users consistently provide negative feedback for certain responses, the data 212 model may learn to avoid those options in the future.

Exemplary data models 212 may include GPT-3 (Generative Pre-trained Transformer 3), BERT (Bidirectional Encoder Representations from Transformers), ROBERTa (Robustly Optimized BERT Approach), ALBERT (A Lite BERT), T5 (Text-to-Text Transfer Transformer), or any other suitable models known to those skilled in the art.

Further, the data model 212 is configured to handle different types of data, including text, audio, image, video and so on. Also, the data model 212 can handle unstructured data. The data model 212 can be a machine learning model and may be configured to process and analyze fed data using techniques such as convolutional neural networks and audio signal processing. The data model 212 may also define the input and output format of the data and how it is processed by the machine learning algorithm.

In summary, data model 212 can handle different types of data, including images and sound, along with text, as long as the structure and relationships between the data is properly defined in the data model 212.

FIG. 3 depicts an example implementation 300 disclosing working of the data model 312 for tracking and management of condition in a patient, in accordance with one implementation of the present disclosure. In comparison to FIG. 2, here the categories under which an appropriate response is generate, in response to received user input, by the data model 312 is shown.

As discussed previously, a data input 302 (such as user input) is received by the system 100 (as shown in FIG. 1). The data input 302 may be related to one or more of sleep tracking 302a, diet tracking 302b, speech tracking 302c, behavior tracking 302d, and intervention tracking 302e. The received input 302 is then received by the data model 312. The data model 312 processes the input 302 to classify the input into one or more relevant condition tracking parameters, based on which the data model 312 generates a response based on the data input 302. More specifically, the data model 312 may first create a user profile 312a and then compares the profile with other user profiles at 312b that are pre-stored, to generate a response appropriate to the at least one user input at 312c. The user profile for each user is updated each time the respective user provides user input including at least one condition related information. The response generated is provided to the user as data output 304, which may include details of relevant doctor/therapists 304a, relevant therapy/medicine 304b relevant diet 304c, or details of relevant community member 304d whose profile matches with the user profile.

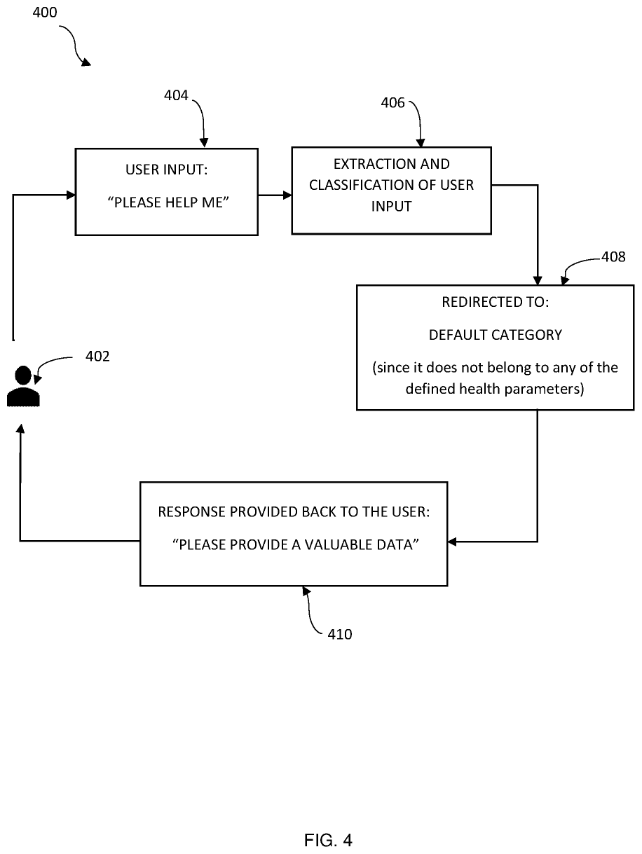

FIG. 4 illustrates an example method 400 for tracking and management of condition in a patient by disclosing use of default category in accordance with one implementation of the present disclosure.

As shown, a user 402 provides a user input 404 via a text-based application such as WhatsApp. Here, the input 404 provided is “PLEASE HELP ME”. The user input 404 is transferred by the communication module (shown as 108 in FIG. 1) to the recommendation generation module (shown as 110 in FIG. 1). The recommendation generating module utilizes the data model (shown as 112 in FIG. 1) to extract and classify the user input 406 on the basis of the already stored condition tracking parameters and identifies that it does not belong to any of the categories (sleep, diet, speech, behavior and intervention). Hence, it automatically classifies and redirects it to a “default category 408”, where the recommendation generation module provides a response 410 to the user “PLEASE PROVIDE A VALUABLE DATA”. The user 402 is then asked for the appropriate data until the time that the user does not provide data related to one or more condition tracking parameters.

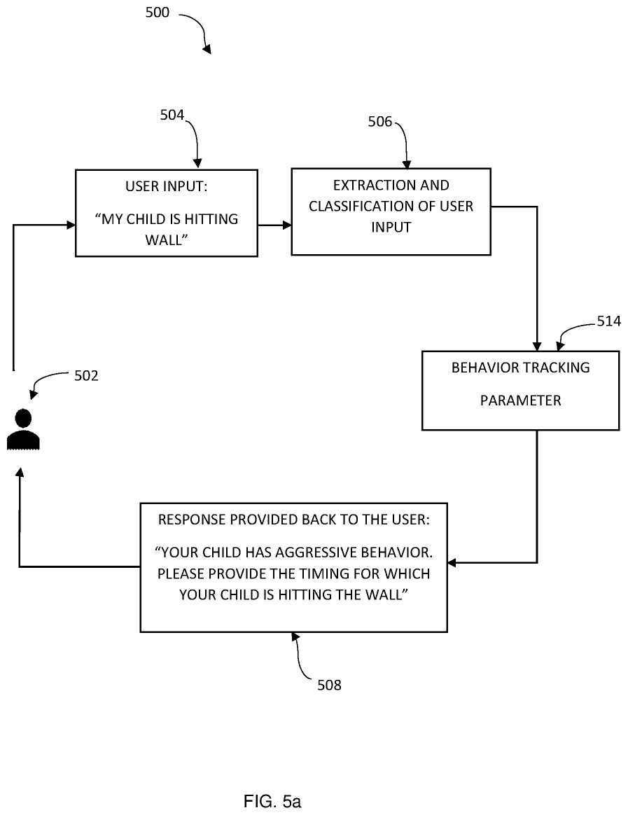

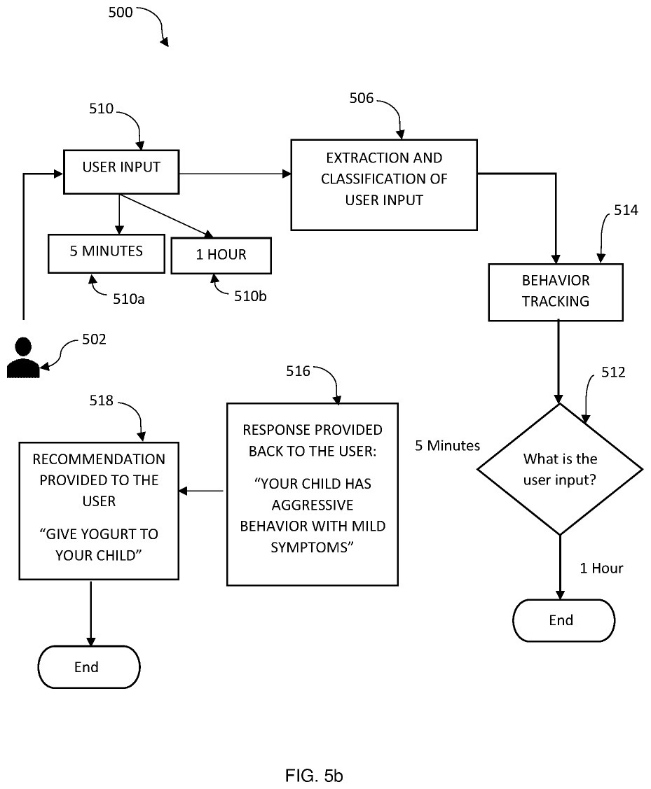

FIGS. 5a and 5b illustrate an example method 500 for tracking behavior 514 in a user 502 and providing an appropriate response for management of condition in a patient, in accordance with one implementation of the present disclosure.

It should be noted that the user 502 who is providing the input may be the patient or a guardian/parent/any other person. It may be understood that the term ‘user’ may be interpreted as a patient, guardian, physician, parent, or any other related person in all other embodiments of this disclosure.

Considering, the user 502 is providing his input 504 (as shown in FIG. 5a) via a text-based application such as WhatsApp. As shown, the input 504 provided is “MY CHILD IS HITTING WALL”. As discussed previously, the user input 504 is extracted and classified at 506 based on the provided user input. Here, the user input 504 is classified under “behavior” tracking parameter 514. A response 508 generated here includes a confirmation message stating “YOUR CHILD HAS AGGRESSIVE BEHAVIOR. PLEASE PROVIDE THE TIMING FOR WHICH YOUR CHILD IS HITTING THE WALL”. The confirmation message is generated based on identification of the intent behind the tracked behavior. The intent may be identified based on further classification of behavior under sub-parameters. For instance, here the intent may be classified under one or more of hyperactive, OCD, self-injurious, aggressive and so on. The user can select the nature of the behavior of the patient based on the mentioned categories or if the user is unable to identify the system automatically classify the behavior of the user based on another user input. In this case, the behavior is classified as “aggressive”. Hence, it will generate the response to the user that “your child has aggressive behavior. Please provide the timing for which your child is hitting the wall”. If the user 502 wants extra information, then he/she may provide another input as well.

Subsequently, in FIG. 5b, as the user 502 is asked for the timing for hitting the wall. Suppose the user provides an input 510 as “5 minutes” 510a, the method continues at block 512, after extraction and classification of the user input at 506, and classify the condition related information into “mild category” and will redirect a response to the user that “your child has aggressive behavior with mild symptoms” 516. While not shown, if the user may provide his input as “1 hour” 510b, an appropriate response is generated. In some case, hitting a wall for 1 hour may classify the behavior under severe aggression. and may redirect a response to the user that “your child has aggressive behavior with severe symptoms”.

Further, the recommendation generating module may also generate a personalized recommendation by comparing the user profile of the user with one or more other user profiles that are pre-stored and matches the user profile. In the shown method 500, the recommendation generated provides a message to the user that “give yogurt to your child” 518, as it helped some other users with the same symptoms. The response mentioned herein is just for the understanding purpose. The user input gets stored in the user profile and the user receive the same response in the form of WhatsApp message (in present example). Although, the response may include text message, push notification, voice response, and video message depending upon the user input.

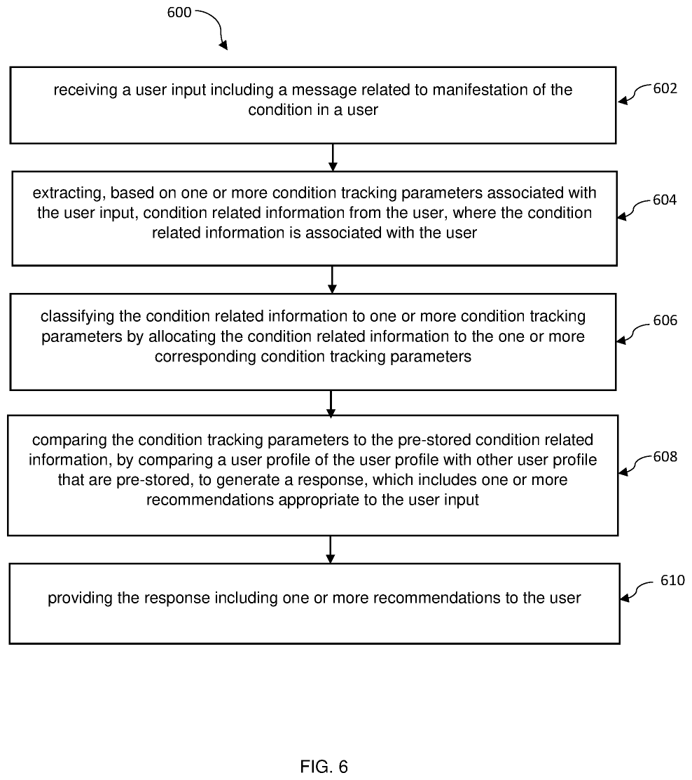

FIG. 6 depicts a method 600 for tracking and management of condition in a patient in accordance with one implementation of the present disclosure.

The method 600 for tracking and management of condition in a patient includes:
Step 602, receiving a user input including a message related to manifestation of the condition in a user;
Step 604, extracting, based on one or more condition tracking parameters, condition related information from the user input, where the condition related information is associated with the user;
Step 606, classifying the condition related information to the one or more condition tracking parameters, by allocating the condition related information to the one or more corresponding condition tracking parameters;
Step 608, comparing the condition related information to pre-stored condition related information, by comparing the user profile of the user with one or more other user profiles that are pre-stored, to generate a response appropriate, which includes one or more recommendations to the at least one user input; and
Step 610, providing the response including one or more recommendations related to management to the user.

The method 600 disclosed here is explained in detail by including the backend processing.

The cloud server (not shown in figure) disclosed in the method 600 above may be Google Cloud, AWS and so on. For instance, consider Google Cloud as server and the user is providing user input using WhatsApp. The API or cloud function are deployed on the cloud server. The Google cloud has more than 150 functions stored in it. A WhatsApp webhook function is generated and is deployed on the Google cloud.

The URL of the WhatsApp webhook function is provided to the Meta developer console. The Meta developer console uses API to transfer the data between the user and the condition tracking application.

When the user provides any input through WhatsApp (in present example), the same data is transferred to the server via Meta developer console. The input data can be in any form, for instance, text, image, audio, video, and so on. Here we are considering the example of WhatsApp so the input data is in the form of text. The user input is transferred to the URL saved in the Meta developer console, which transfers it to the server and as a result activates the function.

There are various AI tools used for processing and comparing the input data received from the user. The AI tools used here may include open AI, API, Google API and so on which classifies the intent diet, speech and sleep into two categories namely valuable and non-valuable.

For instance, if the user enters the message in the WhatsApp and provides user input related to sleep, example, “slept at 9 PM, woke up at 6 AM” or “great sleep last night”. The user input is saved in the memory and a user profile is created mentioning the details of the user input as well as the personal information of the user which may include user phone number, name, email id etc. Further, the user input is transferred to the processing module via communication module. The recommendation generating module further extracts and classifies the user input using data model. Since, the input here is related to sleep intent, the recommendation generating module will extract and classify it into “sleep tracking parameter” using data model. Further, the user profile of the user will be compared to one or more other user profiles that are pre-stored and matches the user profile, to generate a response appropriate to the at least one user input. The recommendation generating module may generate a personalized recommendation based on the comparison of the pre-stored user profile, if the user finds difficulty in sleeping or faces any other issue in which he/she needs help.

In second case, as the data is not provided by user but the user has mentioned that the patient had great sleep, the system will look for the past records and save the data accordingly. The image of the data will be created and sent to the user for his/her reference. This way the transparency of the data is maintained. On the other hand, if the user enters something that is of no use and cannot be categorized, the system will place it under the non-valuable category. It will pop up a message asking the user to enter the appropriate data, till the system classifies it into valuable category.

Similarly, in case of diet intent, the data provided by the user is processed and the system provides protein, vitamin, calories value of that diet to the user. Further, in case of speech intent, the system categorizes valuable content into sound tracking and word tracking. The sound tracking includes 44 sounds, already stored in the database and the word tracking includes joined sounds. Here the input data is provided using Alexa, Siri, Google home or any other audio device. The system classifies the intent behavior into 9 categories namely hyperactive, OCD, self-injurious, aggressive and so on. The user can select the nature of the behavior of the patient based on the mentioned categories.

The API is interacting with the WhatsApp webhook and processing the data.

Further, the classification of input data is done using NLP. The structured data is obtained after processing and is stored in JSON format. The database which is used to store the processed data is FIREBASE.

The processed data sent back to the user may include text messages, push notifications, or voice responses, depending on the design of the system and the input received from the user.

Depending upon the input provided by the user the function varies. For instance, WhatsApp webhook function is used if the user provides input using WhatsApp, TWILIO console is used if the user provides input using SMS and so on.

Advantageously, the system uses deep linking feature to view and the edit the data stored in the App.

This disclosure also helps in providing personalized recommendations based by comparing the user profile with other similarly located profiles. Since there is no biological testing available for autism, different stages can be identified based on observation. In this way, the disclosed system is suitable for predicting the stage of progression of condition and formulates a predictive care plan including the therapies, diet, consultation to the doctors/therapists, consultation with the relevant community member who is facing the similar health related issues and so on that can be beneficial to the patient at a particular stage.

It should be noted that the user profile of each patient is unique and exclusive to the user. The present disclosure uses user profile of a large population of patients to provide an effective way of creating a care plan to the caregivers of the patient suffering from such rare condition. Further, the user profile is updated each time the user provides at least one user input.

In this manner, the system with access to the user profile of various patients improves the ability of the user to successfully access and receive the information and solution regarding autism based on conditions suitable for the patient. The system used in the present disclosure also allows parents and caregivers to prepare personalized recommendations for the autistic patient. The present disclosure also provides a convenient way for users who are trying to retrieve or post details regarding specific episodes of the condition in the patients.

The present disclosure can be implemented on different processing devices configured to communicate with the above discussed condition tracking application. The processing device used here is a processing device which includes a handheld device, a mobile device, a voice enabled device, or a wearable device. In some scenarios, the condition tracking application is installed on the communication device, where the user input user related information in the application either directly or through a bot. In other scenarios, the application is stored on a server configured to communicate with the processing device for receiving the user inputs, where it is not compulsory for the user to install the condition tracking application in the communication device.

As described throughout this application the term “platform” is used to mean a system, application, or any other similar software platform. Also, the terms “parents”, “caregivers”, “users” or “members” will have the similar meaning throughout the disclosure. The condition tracking application of the present disclosure can be installed in any mobile, laptop or computer device. In addition, the system of the present disclosure is relatively inexpensive, safe and easy to use.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

### Technical Advancements

The present disclosure described herein above for tracking and management of condition in patients has several technical advantages including, but not limited to, the realization of:
provides customized care plan based on matching the profile of the user with other similarly located profiles;
suitable for predicting the stage of progression of condition;
user friendly;
easy to use;
does not require installation of apps;
versatility of the chatbot;
maintains transparency of the data;

The embodiments herein and the various features and advantageous details thereof are explained with reference to the non-limiting embodiments in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The foregoing description of the specific embodiments so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

The use of the expression “at least” or “at least one” suggests the use of one or more elements or ingredients or quantities, as the use may be in the embodiment of the disclosure to achieve one or more of the desired objects or results.

Any discussion of documents, acts, materials, devices, articles or the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

The numerical values mentioned for the various physical parameters, dimensions or quantities are only approximations and it is envisaged that the values higher/lower than the numerical values assigned to the parameters, dimensions or quantities fall within the scope of the disclosure, unless there is a statement in the specification specific to the contrary.

While considerable emphasis has been placed herein on the components and component parts of the preferred embodiments, it will be appreciated that many embodiments can be made and that many changes can be made in the preferred embodiments without departing from the principles of the disclosure. These and other changes in the preferred embodiment as well as other embodiments of the disclosure will be apparent to those skilled in the art from the disclosure herein, whereby it is to be distinctly understood that the foregoing descriptive matter is to be interpreted merely as illustrative of the disclosure and not as a limitation.

We claim:

1. A method for condition tracking and management comprising:
receiving a user input including a message related to manifestation of the condition in a user;
extracting, based on one or more condition tracking parameters, condition related information from the user input, wherein the condition related information is associated with the user;
classifying the condition related information to the one or more condition tracking parameters, by allocating the condition related information to the one or more corresponding condition tracking parameters;
comparing the condition related information to pre-stored condition related information, by comparing a user profile of the user with one or more other user profiles that are pre-stored, to generate a response, which includes one or more recommendations appropriate to the at least one user input; and
providing the response including one or more recommendations to the user.

2. The method as claimed in claim 1, further comprising:
creating a user profile including one or more of user information, device information, user input, or generated response, wherein the user profile is updated each time the user provides a new input including at least one condition related information or a new response.

3. The method as claimed in claim 1, further comprising:
including an input provided by an expert appropriate to the user input, wherein the input is used in generating the response and the expert may be a physician or any person having experience in providing recommendations relevant to management of the condition.

4. The method as claimed in claim 1, wherein the response is provided in a format corresponding to the user input, wherein the format of the user input may be one of text input, audio input, video input or image input.

5. The method as claimed in claim 1, wherein the one or more condition tracking parameters include at least one of sleep, diet, behavior, speech, intervention and default.

6. The method as claimed in claim 1, wherein extracting condition related information comprises identifying intent of the message through NLP (natural language processing) of the at least one user input.

7. The method as claimed in claim 1, wherein classifying the condition related information to the condition tracking parameters comprises conducting a contextual analysis of the user input to identify the correlation of the user input to the one or more condition tracking parameters and to identify intention of the user behind the received user input.

8. The method as claimed in claim 1, further comprising
generating a confirmation message, based on contextual analysis of the user input, to receive confirmation of the user on received user input.

9. The method as claimed in claim 1, wherein generating the response including one or more recommendation comprises ranking the recommendations based on effectiveness score, wherein a recommendations having higher effectiveness score is ranked higher as compared to the one or more recommendations having a lower effectiveness score.

10. A computer system for condition management, comprising:
a server including a memory to store instructions; and
a processing device operatively coupled to the server, the processing device to execute the instructions to:
receive a user input, wherein the user input comprises a message related to manifestation of the condition in a user;
extract, based on one or more condition tracking parameters, disease related information from the user input, wherein the disease related information is associated with the user;
classify the condition related information to the one or more condition tracking parameters, by allocating the one or more condition related information to the one or more corresponding condition tracking parameters;
compare the condition related information to pre-stored condition related information, by comparing the user profile of the user with one or more other user profiles that are pre-stored, to generate a response appropriate to the at least one user input; and
provide the response including one or more recommendations to the user.

11. The computer system as claimed in claim 10, wherein the processing device creates a user profile when a first user input is received, wherein the user profile is updated at least each time a new user input is received or a new response is generated.

12. The computer system as claimed in claim 10 further including a condition tracking application, wherein the processing device communicates with the condition tracking application to receive and/or transmit user related information from and to the condition tracking application.

13. The computer system as claimed in claim 10 further including a data model configured to perform a contextual analysis on the received user input to identify an intent behind the user input.

14. The computer system as claimed in claim 13, wherein the data model is a machine learning model configured to process and analyze fed user input using techniques such as convolutional neural networks, audio signal processing, or video signal processing.

15. The computer system as claimed in claim 13, wherein the data model may include one of GPT-3 (Generative Pre-trained Transformer 3), BERT (Bidirectional Encoder Representations from Transformers), ROBERTa (Robustly Optimized BERT Approach), ALBERT (A Lite BERT), and T5 (Text-to-Text Transfer Transformer).

16. The computer system as claimed in claim 10, wherein the user input may be received through one or more user interfaces including a graphical user interface, a voice user interface, a video user interface, a gesture user interface, or any suitable user interface.

17. The computer system as claimed in claim 10 further comprising a user interface, wherein the user provides the user input and receives the response including one or more recommendations via the user interface.

18. The computer system as claimed in claim 10 further comprising a communication module configured to communicate with the condition tracking application to receive and/or transmit user related information.

19. The computer system as claimed in claim 10 further comprising a bot configured to share the received user input with a recommendation generating module, wherein the bot and the recommendation generating module are operatively coupled, and the data model processes the received user input to generate a response corresponding to the received input.

20. The computer system as claimed in claim 10, wherein the processing device may be a handheld device, a mobile device, a voice enabled device, or a wearable device.
